(19) United States
(12) Patent Application Publication
(10) Pub. No.: US 2020/0155053 A1
(43) Pub. Date: May 21, 2020

(54) System and Method for Monitoring and Training Attention Allocation

(71) Applicant: Amit BERNSTEIN, Haifa (IL)

(72) Inventor: Amit BERNSTEIN, Haifa (IL)

(21) Appl. No.: 16/192,027

(22) Filed: Nov. 15, 2018

(51) Int. Cl. A61B 5/16; G09B 5/02; G09B 5/04; G09B 5/06; A61B 5/00

(52) U.S. Cl. CPC A61B 5/168; G09B 5/02; G09B 5/04; A61B 5/748; A61B 5/163; A61B 5/162; G09B 5/06

(57) ABSTRACT

A method and a system for monitoring and training attention real-time attentional bias by applying at least one sensory stimulus over a human subject, using at least one stimulation device, where the sensory stimuli is associated with at least one attentional bias; calculating at least one real-time attention bias score of the subject by measuring response of the subject to the respective applied sensory stimulus; and outputting attentional feedback in real-time indicative of the calculated real-time attentional bias score. The feedback is outputted in real-time or near real-time, using one or more output devices for outputting the attention feedback such as visual or auditory output devices.

### CROSS-REFERENCE TO RELATED PATENT APPLICATIONS

This application is a continuation in-part of U.S. patent application Ser. No. 14/518,498 filed on Oct. 20, 2014, which is a continuation of PCT/IL2013/050342, filed Apr. 18, 2013, which claims priority to U.S. Provisional patent application No. 61/636,121, filed on Apr. 20, 2012, all of which are incorporated herein by reference in their entirety.

### FIELD OF THE INVENTION

The present invention generally relates to systems and methods for monitoring attention allocation of human subjects for improving awareness of subjects to their attention allocation.

### BACKGROUND OF THE INVENTION

Attentional Bias: Attentional bias has been conceptualized and operationalized as preferential allocation of attention to emotion or motivationally-relevant (target) stimuli, relative to competing often emotionally neutral (neutral) stimuli. Though bias is an adaptive capacity to preferentially allocate attention to important events (e.g. danger, appetitive cues), when deregulated, this mechanism drives psychopathology and addictions. The attentional bias literature has predominantly focused on visual spatial selective attention. The large majority of this literature has focused on attentional bias to supraliminal exogenous cues [˜200-1000 ms], such as threat and drug cues. In this context, spatial attentional bias is expressed as facilitated engagement towards target emotional or motivationally-relevant stimuli (e.g., threat, reward stimuli), slower disengagement from those target stimuli, or via attentional avoidance away from those target stimuli. To-date, attentional bias has been calculated via aggregated central tendency statistics (e.g., mean, median) that collapse across multiple estimations or trials measuring (typically spatial but not only) attentional selection and modulation of emotional or motivationally-relevant stimuli/information over time. That is, multiple trial-level observations are recorded repeatedly for tens or hundreds or thousands of trials/observations. Then, typically, the difference score of an aggregated score of observations (e.g., mean or median of reaction time data, eye-movement data) on one sub-set of these trials (e.g., one experimental condition representing ˜50% of all trials) is taken from an aggregated score of observations on a second sub-set of these trials (e.g., a second experimental condition representing the other ˜50% of all trials). This single aggregated difference score represents an aggregated overall trait-like estimation of attentional bias—a trait-like tendency for facilitated engagement towards target emotional or motivationally-relevant stimuli (e.g., threat, reward stimuli), slower disengagement from those target stimuli, or characterized by attentional avoidance away from those target stimuli. From a functional perspective, attentional dyscontrol may contribute to problems with attentional bias. Finally, research suggests that attentional processes underlying attentional bias involve both automatic and strategic processes, and thus may be controllable and modulated to some degree. Consistent with contemporary thinking regarding automaticity and control, these dual process systems are in fact necessarily inter-connected and inter-related. Thus, alleviating attentional dyscontrol may have strong effects on bias as well as on bias-mediated behavioral dysfunction (e.g., psychopathology, addictions).

Attentional Biases & Psychopathology: A well-established body of cross-sectional, longitudinal, experimental, and intervention research has demonstrated that biases of attention represent a core malleable bio-psycho-behavioral risk factor for multiple forms of psychopathology such as anxiety psychopathology and addictions, and related disorders. Greater levels of attentional bias are related to greater levels of psychopathology and various key psychopathogenic processes. Experimental reduction in bias causally results in lower levels of psychopathology and related psychopathogenic processes. This body of research thus provides evidence that attentional bias is a likely causal bio-psycho-behavioral risk factor for a variety of forms of psychopathology and addictions. Thus, attentional bias is one promising focus of the broader, field-wide search for core bio-psycho-behavioral processes underlying the etiology and maintenance of multiple prevalent and often co-occurring forms of psychopathology and addiction.

Attentional Bias Measurement—Behavioral Tasks: Some methodological means to identify attentional bias are well-developed and established. Four primary paradigms have been studied: (1) The visual probe detection task, also described as the dot-probe and visual probe task (see MacLeod, C., Mathews, A., & Tata, P. (1986). Attentional bias in the emotional disorders. Journal of Abnormal Psychology, 95, 15-20); (2) the emotion/addiction stroop (see: Hertel, P. T., & Mathews, A. (2011). Cognitive Bias Modification: Past Perspectives, Current Findings, and Future Applications. Persp. on Psych. Science, 6, 521-536)—both have been studied most extensively; (3) the emotional spatial cueing (see: Fox, E., Russo, R., Bowles, R., & Dutton, K. (2001). Do threatening stimuli draw or hold visual attention in subclinical anxiety? Journal of Experimental Psychology: General, 130, 681-700); and (4) visual search tasks (see: Fox et al, 2001, and Wolfe, J. M. 1994. Guided Search 2.0: A revised model of visual search. Psychon. Bull. & Rev., 1, 202-238).

In the dot-probe task participants are instructed to focus on a centrally presented fixation cross, then stimuli (images, words) are briefly (500 ms) presented at the top and bottom (alternatively, right and left) of the monitor, after stimuli offset a probe appears in location of one of the two stimuli. Participants are asked to indicate the location of the probe by pressing one of two buttons (top vs. bottom/left vs. right). Preferential or biased allocation of attention is inferred from faster response times in congruent trials in which the probe replaces a target cue (e.g., threat), relative to incongruent trials in which the probe replaces the neutral (not target) cue. Thus, as described above [003], in the state-of-the-art, attentional bias has been represented by a single trait-like score based on aggregated central tendency statistics computed across trials, independent of time—representing either facilitated engagement towards target emotional or motivationally-relevant stimuli (e.g., threat, reward stimuli), slower disengagement from those target stimuli, or attentional avoidance away from those target stimuli.

In the (modified) spatial cueing task participants are instructed to focus on a fixation point located between two rectangles, a cue is then presented within one of the rectangles, followed by the brief (e.g., 500 ms) presentation of a target stimulus (e.g., threat cue, drug cue) within one of the rectangles. Participants are instructed to indicate in which of the two rectangles the target stimulus appeared. For “valid” trials, the pre-target cue draws attention to the rectangle in which the subsequent target stimulus will be located; whereas for “invalid” trials, the pre-target cue draws attention away from the rectangle in which the target will be located. Preferential or biased allocation of attention is inferred from faster responses (reduced latency) on valid threat/drug-cued trials relative to neutral-cued trials; as well as slower responses (increased latency) to invalid threat/drug-cued trials relative to neutral-cued trials. Thus, as described above [003], attentional bias has been represented by a single trait-like score based on aggregated central tendency statistics computed across trials, independent of time—representing either facilitated engagement towards target emotional or motivationally-relevant stimuli (e.g., threat, reward stimuli), slower disengagement from those target stimuli, or attentional avoidance away from those target stimuli.

Finally, the (modified) visual search task involves a variety of similar tasks that share the basic feature in which participants are asked to detect a target stimulus (words, images) spatially embedded within a matrix of distracting (e.g., neutral) stimuli; alternatively, a neutral target may be spatially embedded in a matrix of target (e.g., threat, drug) stimuli. This matrix, for example, may be a circle of distracters and target stimuli, or a matrix of rows and columns. Attentional bias is inferred from faster response times to detect a target stimulus in a matrix of neutral stimuli relative to detect a target neutral target stimulus in a neutral matrix; bias is also inferred from slower response times to detect a target neutral stimulus in a matrix of threatening stimuli relative to response times to detect a target neutral stimulus in a matrix of neutral stimuli. Thus, as described above [003], attentional bias is represented by a single trait-like score based on aggregated central tendency statistics computed across trials, independent of time—representing either facilitated engagement towards target emotional or motivationally-relevant stimuli (e.g., threat, reward stimuli), slower disengagement from those target stimuli, or attentional avoidance away from those target stimuli.

Interventions Therapeutically Targeting Attentional Biases: In contrast to our knowledge of attentional biases and their role in psychopathology, the existing knowledge and technological means to systematically affect efficient, large, and lasting change in attentional biases for the purpose of reducing the development and maintenance of multiple forms of psychopathology are, highly limited. Limits of existing knowledge and technology similarly limit means to impact other important adaptive and maladaptive behaviors mediated by attentional bias beyond psychopathology (e.g., food-seeking appetitive behaviors, threat avoidance). Established therapeutic modalities targeting psychopathology, including psychotherapy and pharmacotherapy, have demonstrated mixed, and at best, small to modest therapeutic effects on attentional bias; moreover, these effects likely reflect the effect of reduced psychopathology on attentional bias rather than the effect of reduced attentional bias on psychopathology. This major limitation of extant psycho- and pharmaco-therapies led to the first significant innovation in the clinical science of attentional bias.

Attentional Bias Modification Training: Attentional Bias Modification Training (ABMT), a form of Cognitive Bias Modification Training (which furthermore entails biases in memory and interpretation), reflects the first major clinical science innovation to attempt to target attentional biases. ABMT is grounded in models of implicit conditioning. ABMT implicitly conditions a person's attention away from target stimuli (e.g., threat cues) and towards neutral or positive cues. It does so, for example, by using the dot-probe task, ABMT manipulates the location of the probe to appear exclusively in the spatial location of the neutral cue; this is in contrast to the standard use of the dot-probe task in which the probe randomly occurs in the locations of either the target or neutral stimulus. In short, ABMT conditions participants and patients to look away from target stimuli and towards neutral/competing stimuli on the specific task in which training is delivered. ABMT has been studied most frequently with respect to anxiety, and most commonly social anxiety and generalized anxiety disorders, and bias to threat cues as well as, but less so, with respect to addictive and mood disorders.

Though pioneering, ABMT represents only one very initial and limited means to target attentional bias. First, though ABMT conditions attention away from and towards other cues within a given attentional task, it does not build the capacity to monitor nor self-regulate biased attentional allocation—and thus it does not target the central mechanisms of attentional dyscontrol underlying bias. Second, we lack evidence regarding the generalization of bias reduction conditioned within a specific paradigm (e.g., dot-probe) to other paradigms (e.g., visual search) for which no implicit conditioning was delivered—calling into question whether the bias is extinguished beyond the conditioning paradigm. Third, there is very limited evidence of durable (over-time) bias reduction; notably, bias reinstatement effects are likely as implicit conditioning results in highly context-specific extinction of bias. Fourth, the magnitude or size of effects of ABMT on attentional bias are typically small. Thus, the clinical significance of ABMT may ultimately prove to be relatively modest. Fifth, implicit conditioning attention away from a given target (e.g., threat) cue may in fact be contraindicated to achieve its long-term therapeutic aims. ABMT may incidentally promote a form of maladaptive attentional avoidance. Because attentional avoidance is thought to maintain anxiety psychopathology via limiting adaptive, elaborative processing of feared stimuli and its salutary processes (e.g., reappraisal), it may maintain or strengthen associations with threat or drug cues. Finally, ABMT scholars make clear that there is no theoretical argument, empirical evidence, nor neurobiological rationale that ABMT is the only means, necessarily the optimal, nor most effective means to reduce attentional bias.

US patent application No. 2011/0105937 by Pradeep et al, discloses a system that analyzes controlled and automatic attention for introducing stimulus materials (mainly media materials such as video, audio etc.). This system analyzes neuro-response measurements (such as electroencephalography (EEG)/event-related potential (ERP) based measurements) from subjects who are exposed to stimulation that elicit controlled and automatic attention. The purpose of this system is to identify location over the media that is associated with “high controlled attention metrics” and placing introduction stimulus materials in those locations. Another US patent application No. 2009/0327068 by Pradeep et al discusses a similar system that allows performing stimulus targeting using neuro-psychological and neuro-behavioral data taken from various available measuring systems and methods such as EEG/ERP, Galvanic Skin Response (GSR) etc.

There are patents and patent applications that discuss tracking of subjects' responses to various visual or other sensory stimuli for analyzing various psychological and/or other characteristics of the subjects, such as a patent application No. WO2007/040857 by Ghajar Jamshid. Ghajar discloses a system for testing cognitive impairments of subjects by providing the subject with multiple stimuli including a smoothly moving object, while tracking the subject's eye-movements. An analysis of the movements allows determining if the subject has a cognitive impairment.

Other patents and applications use stimuli-response analysis for improving image analysis and/or presentation such as US patent application No. 2011/206283, by Quarfordt et al, which teaches a method and a system for improving image analysis using gaze-data feedback; and/or U.S. Pat. No. 7,822,783, which discloses a method and apparatus for real time adaptation of presentation to individuals or US patent application no. 2009/0024049, disclosing combining a multiplicity of modalities of responses to stimuli of the nervous system.

US patent application No. 2008/0275358 by Freer et al teaches a training method for employing brainwave monitoring. According to Freer, a brainwave monitor is employed for determining level of attention and providing a training environment, in which the trainee is provided with a feedback indicating to the trainee whether he/she is in focus or not, while providing the trainee with an incentive to stay focused. In this case, the degree to which the subject is focused is measured and not the attention allocation of the trainee or degree to which their allocation is biased or preferential to certain cues or predefined stimuli.

Research by Rothermund (Rothermund, Klaus, “Motivation and Attention: Incongruent Effects of Feedback on the Processing of Valence”, Emotion, Vol3(3), Sep. 2003, 223-238) tested the influence of feedback (inducing related motivational state of a subject) on evaluative decision. In this research, each subject was given positive/negative task-performances feedback (such as success or failure feedbacks) for inducing his/her motivational state in respect to each exercise/task for measuring his/her following performances in a valence related task. In Rothermund's experiments only task-performance feedback was given for the purpose of manipulating the motivation of the subject in order to check the subject's subsequent attention allocation to valence of stimuli such as a word that is either congruent or incongruent in valence with the induced motivational state.

Dibartolo (Dibartolo, Patricia Marten, “Effects of Anxiety on Attentional Allocation and Task Performance: an Information Processing analysis using Non-Anxious and Generalized Anxiety Disorder Subjects”, Dissertation Abstracts International: Section B: The Sciences and Engineering. Aug. 1996, pp. 1435) studied attentional allocation and its influence on normal comparison (NC) subjects and subjects who suffer from generalized anxiety disorder (GAD). In particular the impact of neutral distractor and negative feedback cues on performance of an attention vigilance task was investigated. Individuals with GAD evidenced impaired performance on an attention vigilance task relative to NC subjects when neutral distractor cues were presented. Contrary to prediction, no group differences in performance were detected under conditions in which subjects were presented negative feedback cues they were told were relevant to their performance. Instead, GAD participants exhibited improvement during the experimental task such that their performance was equivalent to NC subjects.

A US patent application No. 2011/027765 by Nader Amir discloses systems for treating patients with an anxiety disorder that comprise a screen for displaying sets of stimuli, a computer to control the display of stimuli onto the screen during at least one treatment session and the ability for the patient to interact with the screen in response to the displayed stimuli. The interaction of the patient with the system during the treatment session is capable of treating patient anxiety associated with an anxiety disorder, such as social anxiety. The interaction includes, for example responding to other stimulations of interactivity appearing on the screen or interacting with a human therapist.

International patent application WO 2003/075762 by Lilach Shalev discloses a method and system for diagnosis and treatment of Attention Deficit Hyperactivity Disorder (ADHD) or Attention Deficit Disorder (ADD). It is designed to diagnose ADHD/ADD by evaluating individual scores on a variety of established cognitive tasks designed to measure cognitive and attentional tasks. It is designed to train and improve attentional functions in ADHD by exposing a subject to various “cold cognition” cognitive tasks (e.g., related to alerting, sustained attention, etc.) and then training the subject by making those tasks incrementally more difficult thereby requiring and motivating subjects to continuously improve performance on these tasks. In addition, to motivate subjects to remain engaged with the cognitive tasks and to reward their performance, the system provides performance feedback on a “plurality of trials” (e.g., on aggregated scores that collapse across performance over long periods of performance) and on a “tight schedule” (e.g., on aggregated scores that collapse across shorter periods of performance)—feedback to the subject indicating whether participants' performance across many observations/trials of task improved or whether their responses to task trials or a plurality of trials was “correct” or “incorrect”.

### SUMMARY OF THE INVENTION

It is an object of the present invention to provide a system and method for outputting attentional bias feedback.

It is another object of the present invention to provide a system and method for outputting real-time attentional bias feedback.

It is a further object of the present invention to provide a system and method for outputting real-time attentional bias feedback for training.

The present invention relates to a computing system comprising at least one processor; and at least one memory communicatively coupled to the at least one processor comprising computer-readable instructions that when executed by the at least one processor cause the computing system to implement a method of outputting a real-time attentional bias feedback. The method comprises:

a) applying, using at least one stimulation device, at least one sensory stimulus at time N over a human subject, the at least one sensory stimulus being associated with at least one attentional bias, wherein the at least one attentional bias represents impaired attentional engagement with or disengagement from the at least one sensory stimulus;

b) using at least one measuring device that measures physiological responses to the respective stimulus, measuring at least one response of the human subject to the applied at least one sensory stimulus at time N;

c) calculating at least one real-time attentional bias score of the subject at time N, wherein the at least one attentional bias score is calculated based on: (1) the at least one measured response of the subject to the at least one applied sensory stimulus at time N; and (2) at least one temporally preceding measured response of the subject to the at least one applied sensory stimulus; and

d) using at least one output device, outputting in real-time the real-time attentional bias feedback at time N indicative of the calculated real-time attentional bias score at time N, the real-time attentional bias feedback being output relative to a corresponding attentional bias scale.

In some embodiments, after steps a) to d) are executed initially, the system repeats steps b) to d) one or more times.

In some embodiments, after steps a) to d) are executed initially, the system repeats steps a) to d) one or more times.

In some embodiments, the at least one temporally preceding measured response comprises a temporally preceding adjacent measured response.

In some embodiments, the at least one sensory stimulus comprises at least one of: visual stimulation, auditory stimulation, tactile stimulation, olfactory stimulation, and/or gustatory stimulation.

In some embodiments, the measured response in step b) comprises at least one of: reaction time indicative of the time it took the subject to respond to the at least one applied stimulus; task time, indicative of the time it took the subject to fulfill a requested task associated with the respective applied stimulus; or at least one physiological measure indicative of physical response of the subject to the stimulus.

In some embodiments, the measuring response in step b) is carried out by using at least one predefined attention interference scheme, and wherein the scheme is based on at least one of: a dot-probe paradigm; a spatial cueing paradigm; a visual search paradigm; and/or modified stroop task.

In some embodiments, the measuring response in step b) is carried out by using an eye tracking system that measures the subject responses to visual stimuli.

In some embodiments, the measuring device comprises one of: an eye-tracking system, a magnetic resonance imaging (MRI) device, a psychophysiological device, an electroencephalography (EEG) or an event-related potential (ERP) device.

In some embodiments, the real-time attentional bias feedback is a visual feedback, auditory feedback, tactile feedback or any combination thereof.

In some embodiments, the visual feedback includes at least one of: a graphical indication representing statistics of the bias level or related nature of the attentional bias of the subject and/or of multiple subjects in relation to some or all previously measured responses; a graphical indication of the attentional bias of the subject in relation to the last stimulus.

In some embodiments, the system further comprises at least one input device for allowing the human subject to respond to said sensory stimulus therethrough, wherein said input device includes at least one of: a touch screen, a computer mouse, and a keypad.

In some embodiments, the outputting real-time attentional bias feedback trains attention allocation of the human subject.

In another aspect, the present invention relates to a computing system comprising at least one processor; and at least one memory communicatively coupled to the at least one processor comprising computer-readable instructions that when executed by the at least one processor cause the computing system to implement a method of outputting a real-time attentional bias feedback. The method comprises:

a) applying, using at least one stimulation device, at least one sensory stimulus at time N over a human subject, the at least one sensory stimulus being associated with at least one attentional bias, wherein the at least one attentional bias represents impaired attentional engagement with or disengagement from the at least one sensory stimulus;

b) using at least one measuring device that measures physiological responses to the respective stimulus, measuring at least one response of the human subject to the at least one applied sensory stimulus at time N;

c) calculating at least one real-time attentional bias score of the subject at time N, wherein the at least one attentional bias score is calculated based on the at least one measured response of the subject to the at least one applied sensory stimulus at time N; and

d) using at least one output device, outputting in real-time an attentional feedback at time N indicative of the calculated real-time attentional bias score at time N, the attentional feedback being output relative to a corresponding attentional bias scale.

In another aspect, the present invention relates to a computing system comprising: at least one processor; and at least one memory communicatively coupled to the at least one processor; at least one stimulation device configured to apply at least one sensory stimulus at time N over a human subject, the at least one sensory stimulus being associated with at least one attentional bias, wherein the at least one attentional bias represents impaired attentional engagement with or disengagement from a sensory stimulus; at least one measuring device configured to measure at least one response of the human subject to the applied at least one sensory stimulus at time N; and at least one output device configured to output a real-time attentional bias feedback at time N, wherein the at least one processor is configured to calculate at least one real-time attentional bias score of the subject at time N, wherein the at least one real-time attentional bias score is calculated based on: (1) the at least one measured response of the subject to said applied at least one sensory stimulus at time N; and (2) at least one temporally preceding measured response of the subject to the applied at least one sensory stimulus; and wherein the real-time attentional bias feedback is indicative of the calculated real-time attentional bias score at time N, said real-time attentional bias feedback being output relative to a corresponding attentional bias scale.

### BRIEF DESCRIPTION OF THE DRAWINGS

FIG. 1 is a flowchart, schematically illustrating a method for real-time output of attentional feedback according to some embodiments of the present invention.

FIG. 2 is a flowchart schematically illustrating an embodiment of a process of real-time output of attentional feedback, according to one embodiment of the present invention.

FIGS. 3A-3D show four successive screenshots representing a process of outputting real-time attentional feedback after applying a visual stimulus associated with an attentional bias, by using a dot-probe based visual stimuli, according to one embodiment of the present invention: FIG. 3A shows a gaze neutralizing screenshot; FIG. 3B shows a screenshot in which a target and neutral images are presented, according to a dot-probe based monitoring and stimulating technique; FIG. 3C shows a screenshot in which a probe is shown replacing the target image of the screenshot of FIG. 3B; and FIG. 3D shows a screenshot in which the real-time attentional feedback of the subject's real-time attention bias in the respective task are visually represented in real time.

FIG. 4 shows some of the steps of the process of FIG. 2 showing how the attentional feedback can be presented through an attentional bias scale indicator showing a dynamic moment-to-moment or trial-to-trial attention “bias level” of the subject in real-time, according to one embodiment of the present invention.

FIG. 5 schematically illustrates a system for real-time output of attentional feedback, according to some embodiments of the present invention.

FIG. 6 schematically illustrates a system for real-time output of attentional feedback, according to additional or alternative embodiments of the present invention.

FIG. 7 shows experimental results for measuring physiological responses of subjects who were provided real-time feedback (A-FACT) on their dynamic moment-to-moment or trial-to-trial real-time attention bias relative to subjects who were not provided with such sensory stimuli (sham placebo control), according to one embodiment of the present invention.

### DETAILED DESCRIPTION OF THE INVENTION

In the following detailed description of various embodiments, reference is made to the accompanying drawings that form a part thereof, and in which are shown by way of illustration specific embodiments in which the invention may be practiced. It is understood that other embodiments may be utilized, and structural changes may be made without departing from the scope of the present invention.

The present invention, in some embodiments thereof, provides methods and systems for outputting real-time attentional feedback to a human subject indicative of a calculated real-time attentional bias score.

First, using at least one stimulation device, one or more sensory stimuli are applied to human subject at time N. The sensory stimuli are associated with at least one attentional bias of that human subject. An attentional bias represents impaired attentional engagement or disengagement with a sensory stimulus.

At time N, the response of the human subject to the applied sensory stimulus is measured using an adapted measuring device that measures physiological responses to the respective stimulus.

The real-time attentional bias score is calculated based on: the measured response of the subject to the applied sensory stimulus at time N relative to at least one temporally preceding (but need not be adjacent) measured response of the subject to the applied sensory stimulus.

One advantage of the invention for a human subject is that by using the system he is able to improve his/her awareness of, and thereby achieve cognitive control over, his/her momentary biased attentional processing of emotion or motivationally-relevant information attention. These novel methods and systems may be used for real-time monitoring of at least one attentional bias and providing real-time attentional feedback that is associated therewith, for allowing subjects to develop and practice their awareness of their dynamic moment-to-moment or trial-to-trial biased attentional processing of emotion or motivationally-relevant information or stimuli, and thereby gaining cognitive control over their biased attentional processing of this information and their related responding to target stimuli associated with their attentional bias.

Targeting awareness of biased attentional processing of emotional or motivationally-relevant information by means of real-time attentional feedback on the calculated real-time attentional bias score may improve subjects' capacity to self-monitor their moment-to-moment dynamic expression of attentional bias and behavioral correlated associated therewith, and thereby increase subjects' regulation and control of their biased attention. Improving control over one's responses to cues of stimuli associated with specific attentional bias(es) may be a powerful training tool for helping subjects to neutralize pathogenic effects of attentional bias on the development and maintenance of multiple forms of psychopathology and addictions or potentially prevent such psychopathologies from occurring. Training awareness and control of biased attention via real-time attentional feedback may also have a variety of other therapeutic benefits such as reduction of maladaptive behaviors and promotion of adaptive behaviors that are closely linked to attentional bias such as safety-related behaviors including, for instance, security-related behaviors (e.g. improving attention allocation to threat related cues), driving, flight, combat behaviors and the like, and/or weight loss or drug seeking/quitting related behaviors.

Real-time attentional feedback means that substantially immediately after the stimulus is applied (typically less than one second), the calculation of attentional bias substantially immediately follows the measured response on trial N and the delivery of feedback on that attentional bias substantially immediately (under one second) following that real-time calculation on trial N.

Training awareness and control of momentary expressions of biased attention via real-time attentional feedback can also be used as a tool to train a human subject to reduce, acquire, or strengthen attentional biases for improving their instinctive responses to specific cues associated with those specific biases. This can be used, for example, for training law enforcement professionals to improve their attentional allocation to cues related to selected biases such as potential threat. Other similar applications include behaviors that mediated by attentional such as a wide range of appetitive behaviors (e.g., food-seeking), driving, flight, or security/combat.

According to some embodiments of the present invention, the human subject is given a set of tasks to perform. In each task, one or more stimuli are applied on or presented to the subject such as visual, auditory, olfactory and/or tactile stimulus, through one or more stimulation devices such as a screen, a speaker, a light emitting source, vibrating devices for tactile stimulation, and the like. Each sensory stimulus is associated with at least one attentional bias.

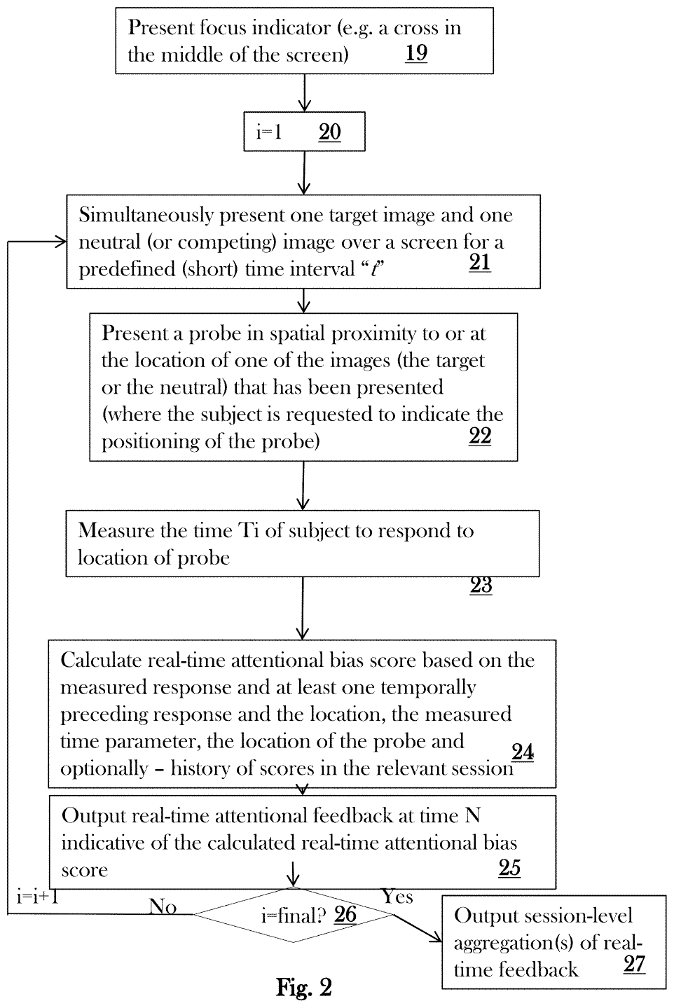

Visual stimulation device—a screen, such as a computer monitor, or a television displays any combination of still images and video for predetermined durations on predetermined areas of the screen as further described in FIG. 2.

Auditory stimulation device—any speaker connected to an audio source (computer, amplifier etc.) and outputting predetermined sounds at predetermined timings.

Tactile stimulation device—any vibrating device that is touching the user (or very close to the user) and which vibrates at predetermined vibration strength and frequency, at predetermined timings.

The subject's response to the stimuli are then measured and recorded (e.g. by measuring the time it took the subject to divert his/her gaze from a target image to a reference point or the subject's input response to a provided task). At least some of the stimuli applied on the subject is associated with attentional biases that are related to one or more predefined areas such as threat cues (e.g. image, word or sound that are related to threat) or appetitive cues.

Based on the measured response (or responses) of the subject to the sensory stimuli, the system calculates in real-time at least one attentional bias score. There can be different ways to calculate an attentional bias score, but all these real-time calculations at time N, use the measured response (or responses) at time N together relative to at least one temporally preceding (but not necessarily adjacent) measured response of the subject.

The measurements that are used to calculate in real-time the attentional bias score may include any one of the aforementioned paradigms such as the dot-probe paradigm, the emotional spatial cueing paradigm or the visual search-based paradigm. Any type of known in the art technique, system or device may be used to apply the stimuli and/or to acquire the measured response (or responses) such as eye-tracking systems, sound systems, systems that can stimulate and/or measure biological responses of the subject such as psychophysiological (e.g., skin conductance), electroencephalography (EEG/ERP), magnetic resonance imaging (MRI) based systems and the like.

The real time attentional feedback may be indicative of any one or more of the measured responses. The attentional feedback may take any form such as visual, auditory, tactile, or any combination thereof, reflecting one or more values of one or more calculated attentional bias scores on recent trial(s) or task(s).

The systems and methods of the present invention can be designed especially to address one or more predefined areas or fields that cause at least one attentional bias (defined hereinafter as “bias fields”) such as threat, reward, or any other field that is often associated with attentional biases in the general population, specific subpopulation(s), or in unique individuals.

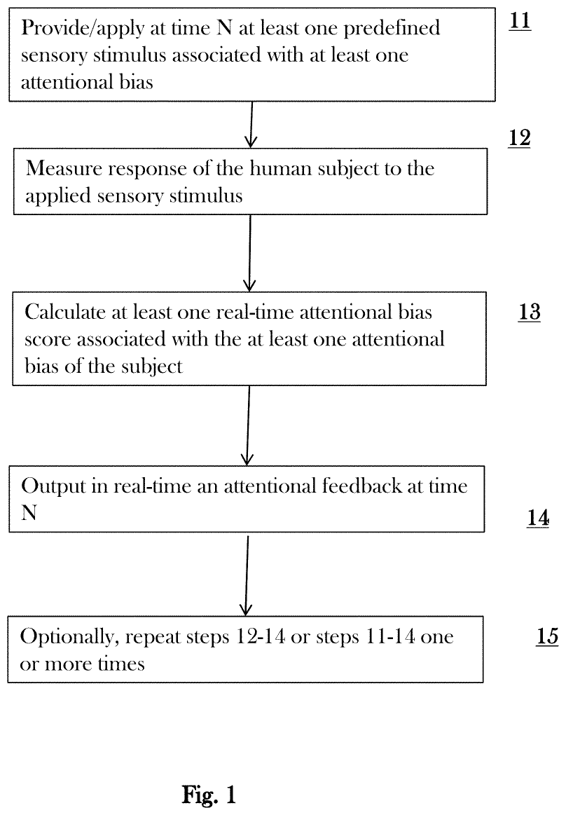

Reference is now made to FIG. 1, which is a flowchart, schematically illustrating a computerized method for outputting real-time attentional feedback related to a specific predefined attentional bias, according to some embodiments of the present invention. The method includes: (i) in step 11, applying at time N a sensory stimulus associated with one or more specific and predefined form or expression of attentional bias (e.g. by using visual and/or auditory predefined cues associated with these attentional bias(es)); (ii) in step 12, measure response (or responses) of the subject to the provided stimulus, for example, measuring the subject's behavioral and/or physical response to the stimulus (e.g. by detecting responses such as eye movements, brain electrical activity, and the like or by receiving response input by the subject); (iii) in step 13, calculating in real-time at least one attentional bias score associated with at least one attentional bias of the subject. The attentional bias score is calculated based on the measured response of the subject to the applied sensory stimulus at time N; and at least one temporally preceding measured response of the subject to the applied sensory stimulus; and (iv) in step 14, outputting (e.g. presenting) in real-time attentional feedback indicative of the calculated attentional bias score such as the values of one or more real-time attention bias scores of the subject. In step 15, steps 12-14 or 11-14 can be repeated one or more times. For example, after initial execution of steps 11-14, steps 12-14 may be executed once or many times per second or once every several seconds and then steps another sensory stimulus (step 11) may be applied and steps 12-14 may be executed again one or more times.

At least some of the attentional feedback associated with the respective stimulus will be provided to the subject substantially immediately following her/his measured response to trial N stimulus and before the next stimulus (trial N+1) is applied. Therefore, the attentional feedback associated with the specific stimulus at time N is delivered substantially immediately (meaning in one second or less than one second) following calculating the real-time attentional bias score associated with the provided stimulus at trial N.

The measured response (or response) in step 12 may include (i) response time indicative of the subject's physiological or behavioral time of response to the applied stimulus(i) at trial N; (ii) task time of the subject at trial N; (iii) task performance of the subject in response to the applied stimulus(i); (iv) physiological measures of the subject in response to the applied stimulus(i) such as electrical brain activity; and/or any other measurable physiological and/or behavioral measures indicative of the subject's attention selection and modulation in response to the applied stimulus(i).

This process (steps 11-15) may be carried out using special software and/or hardware modules operated through one or more computers that allow outputting visual and/or auditory stimuli (such as presenting words or images over the computer screen of target and neutral cues as in the dot-probe paradigm) through devices of the computer such as the screen and/or speaker thereof. In this particular interference scheme task (dot probe), once the target and neutral stimuli are simultaneously presented, the module either requires the subject to actively respond to the stimuli by requiring him/her to indicate the location or features of one of the stimulus cues presented or passive measurement of physiological response of the subject to the trial stimulus using response measuring equipment such as an eye-tracking system, a magnetic resonance imaging (MRI) based system and the like. The response parameter(s) (such as the response time, eye-movement, electrophysiologic response) that is (are) recorded by the respective technology/methodology of the system and the trial-level real-time attentional bias score is calculated and then presented to the subject in real-time to provide the subject real-time or near real-time (substantially immediate) attentional feedback in response to the applied stimulus so as to train a subject's awareness of and control over his/her biased attentional processing of that stimulus in that moment. The proposed system thus creates two entirely novel functions that were not capable of existing technologies used to constitute to the novel system. First, existing technology was not able to calculate attentional bias in real-time from moment to moment, but only to calculate an aggregated estimate of attentional bias based on performance across a “plurality of trials” substantially following multiple expressions of attentional bias (e.g., aggregated mean/median bias scores that collapse across performance over long periods of repeated measured responses of attention such as following an entire block of hundreds of trials). Consequently, second, existing technology was not able to deliver real-time feedback on attentional bias and thereby to train awareness and control, critically, concurrent with the moment-to-moment expression of attentional selection and modulation. Instead, prior to this invention, existing technologies could only provide performance feedback on a “plurality of trials” substantially following multiple expressions of attentional bias (e.g., on aggregated scores that collapse across performance over long periods of performance such as following an entire block of hundreds of trials).

According to some embodiments of the invention, as mentioned above, when using visual stimuli, e.g. in any one of the above-mentioned paradigms through which we calculate in real-time an attentional bias score of the subject in relation to a specific bias field, eye-tracking can be used to track the subject's overt attentional processing of that emotional or motivationally-relevant stimulus(i) or information. Eye-tracking of eye-movement can use simultaneous tracking of both the center of the pupil location and the corneal reflection, which together allow computation of gaze direction parameter. Temporally eye movements can be broken down into movements of the fovea on the visual field (saccades) and periods of relative stability during which an object can be viewed (fixations). Eye-tracking may provide a more direct indicator of overt visual attention and therefore of attention allocation relative to behavioral reaction-time. Moreover, eye-tracking permits measurement of attention allocation in a temporally continuous manner—measuring components (engagement, disengagement, avoidance) of attentional allocation in real-time, rather than via more temporally distal and gross behavioral indices (such as the user's active pressing over a key indicating the location of the probe as in the dot-probe monitoring paradigm). Due to the invention and proposed system, eye-tracking technology is given two entirely novel functions that were not capable of eye-tracking technology to-date. First, existing eye-tracking technology was not able to calculate attentional bias in real-time from moment to moment, but only to calculate an aggregated estimate of attentional bias based on eye-movement performance across a “plurality of trials” substantially following multiple expressions of attentional bias based on eye-movement (e.g., aggregated mean/median bias scores that collapse across eye-movement performance over long periods of repeated measured eye-movements such as following an entire block of hundreds of trials). Consequently, second, existing technology was not able to deliver real-time feedback on attentional bias based on those eye-movements and thereby to train awareness and control, critically, concurrent with the moment-to-moment expression of attentional selection and modulation. Instead, prior to this invention, existing technologies could only provide performance feedback on a “plurality of trials” substantially following multiple expressions of attentional bias (e.g., on aggregated scores that collapse across eye-movement performance over long periods of repeated eye-movement responses such as following an entire block of hundreds of trials).

Reference is now made to FIG. 2, which is a flowchart schematically illustrating a process of real-time calculation of attentional bias and real-time attentional feedback presentation of the subject's real-time attentional bias at trial N, using the dot-probe paradigm for calculating in real-time the subject's attentional bias score, according to one embodiment of the present invention. The process includes a set of a predefined number of tasks exercises each allows measuring response time of the user to a visual stimulus. Once the process is initiated 20, at each respective task “i”, two images are simultaneously presented to the subject 21 (e.g. over a computer screen) for a predefined short time interval “t”: a target image and a neutral (or competing) image e.g. representing target and neutral cue pictures/words respectively. A probe (or any other type of visual indicator) is then presented over the screen 22 at the location or in proximity to the location of one of the images (the target or the neutral) depending on the task definition (performing congruent or incongruent trials as described above). The subject is either requested as part of the session instructions to point the location of the probe or the identification of the probe location is carried out automatically (e.g. by using eye-tracking systems and techniques). The response time Ti is then measured 23 by measuring the time it took the subject to identify and input the location/feature of the probe. The response time Ti may be immediately presented over the monitor for providing the subject with a real-time feedback of his/her performance at trial N 25.

Additionally, or alternatively, once each response time Ti is measured 23, a real-time attentional bias score may be calculated 24 and presented as real-time feedback 25.

Real-time calculations at time N use the measured response (or responses) Ti at time N together with at least one temporally preceding (but not necessarily adjacent) measured response. There are several instantiations of this type of “N−1” calculation 24 to enable real-time feedback 25. For any given target trial N, the temporally contiguous preceding empirical reference or comparator may be a response(s) on a preceding temporally continuous trial, some fixed value estimated at some point prior to the target trial, or the value(s) of the response(s) on an updating, running window which may produce for example an updating or “running” central tendency statistic(s) (e.g., mean, median) that were temporally contiguous and preceded the target trial for which we are estimating trial-level real-time attentional bias. A person skilled in the art reading the current disclosure, will understand immediately how to calculate the real-time attentional bias score and output the real-time attentional bias feedback using the proposed system. Provided below are examples of possible calculations and methodologies:

### Example 1—Trial-Level Real-Time Attention Bias Calculations

We can match and then subtract temporally contiguous pairs of trials, one trial is the target trial at trial (or time) N and we compare and subtract it from its temporally contiguous immediately preceding comparator trial (or time N−1). The target trial is one wherein attentional bias may be observed and for which we need to calculate a trial-level or real-time attention bias score at time N. The comparator trial is the trial, that is the preceding, most temporally contiguous, trial wherein the same response (e.g., reaction time, eye movement, electropohysiologic signal) was measured but wherein no or a different attentional bias could have been observed. For example, the comparator trial (N−1) may be an emotionally neutral trial, or for example in the context of common attentional bias interference schemes that include incongruent and congruent trials or invalid and valid trials or predictive or non-predictive trials, whatever the trial type at trial N is, we match the measured response(s) on that trial N to the measured response(s) on the N−1 alternative or complementary trial type that preceded it (i.e., the comparator trial). In terms of operationalizing temporal contiguity, this is variable and is determined by a number of factors such as the number of trial types, the timing (e.g., 1-trial/sec vs. 10-trials/sec), but will always find the preceding comparator trial that is as temporally contiguous as possible so as to optimally estimate attention bias at trial N relative to that temporally contiguous comparator.

For example, each congruent trial (CT) or incongruent trial (IT) may be matched with a neutral comparator trial (NT) that is temporally as close as possible following the target CT/IT (i.e., computed as IT-NT and NT-CT). This may be done with one or more response types per trial. For example, this may be concurrently done with covert attention indices such as reaction time as well as overt attention indices such as eye movement on the same series of paired trials to estimate real-time attentional bias.

### Example 2—Trial-Level Real-Time Attention Bias Calculations

In another instantiation of this calculation approach to estimate trial-level real-time attentional bias, we cannot only compare response (or responses) on a target trial at time/trial N to a temporally contiguous preceding comparator trial but to some “fixed” comparator reference value of the measured response(s) of interest (e.g., such a value may reflect “no bias” or some other comparator value of interest). Then, that fixed comparator reference value is then repeatedly contrasted, at the trial-level in real-time with the response(s) on each target trial.

### Example 3—Trial-Level Real-Time Attention Bias Calculations

In yet another instantiation of this calculation, we can also compare response(s) on a target trial at time/trial N to an updating calculated comparator reference based on multiple comparator trials such as via a running window of responses on multiple temporally contiguous comparator trials for which the response(s) of interest is measured. Thus, for any given target trial N, the temporally contiguous preceding empirical reference or comparator may be the value(s) of the response(s) on an “running” or updating window of trial responses which may produce, for example, a running central tendency statistic(s) (e.g., mean, median) and or statistic(s) of variability (e.g., standard deviation) of responses in that updating window of multiple trails that were temporally contiguous and preceded the target trial N for which we are estimating trial-level real-time attentional bias. In this instantiation of the calculation, the comparator reference will reflect multiple repeated and updating observations of the comparator response(s) but will still reflect attentional processing and respective measured responses that are updated in time and temporally preceding the target trial N for which real-time attentional bias is calculated and real-time feedback is delivered.

For example, using an updating or running mean (multiple trials window temporally contiguous and preceding each target trial) and confidence interval (95% CI) were calculated for comparator neutral trials, and then (linearly or nonlinearly) interpolated trials. We then compare and contrast the measured response(s) on each target trial to comparator 95% CI of the running window, repeatedly for each target N and for the updating running window and updating 95% CI of that window of comparator trials.

Alternatively, real-time calculations at time N may use the measured response (or response) at time N alone without direct comparison to temporally preceding measurement. There are a number of instantiations of this type of “N” calculation.

For example, there are methodological schemes in which responses at time N—if N is a single trial or if there are multiple N time-points within a single trial—are sufficient to estimate real-time attention bias and thereby deliver real-time feedback. These include, for example, calculations wherein the comparator reference is presented concurrent with the target stimulus and responding to the target (e.g., eye-movement) also directly reflects concurrent responding to the comparator stimulus and thus no comparison to an earlier N−1 trial/reference value(s) is needed to estimate trial-level real-time attentional bias. A common instantiation of this calculation of real-time attentional bias may be observed on various “free view”-type stimulus presentations that are well-known in the art.

Pointing out the location of the probe 22 (which is one of two location choices in the dot-probe based training such as up/down or left/right etc.) may be carried out by using a specific predefined marking/indicating technique such as using the computer mouse to bring the cursor to the location of the probe; using automatic eye-tracking technique; pressing one predefined key to indicate that the probe was in one optional location and another key for indicating that the probe was at the other optional location; using audio input (saying the position of the probe); and the like.

Optionally, as illustrated in FIG. 2, a neutralizing stimulus may be provided 19 at the beginning of the entire process or set (block) of trials (also referred to as session) or before each trial task “i”. The neutralizing stimulus may include presenting a cross at the middle of the screen for neutralizing the subject's gaze before each trial N task i or before each session.

The time interval “t” may be constant or vary (e.g. decrease or increase) according to a predefined setup or according to the resulting measurements of the measured response time “T”.

Following repeated delivery of real-time feedback for each trial N 26 base on each calculated real-time attentional bias score for each trial N 25, an additional form of post-session feedback (cf. the real-time trial-level feedback 26) may be presented to the subject 27, indicative of some aggregation of the subject's real-time attentional bias scores across the session or earlier trials. This parameter(s) may reflect a number of aggregated values related to the measured attentional bias over the course of the session and repeated trails, such as a central tendency statistic representing those values, a value representing observed pattern of those repeated real-time attentional bias scores, a value representing temporal variability or stability of those repeated real-time attentional bias scores, or a value(s) reflecting degree and direction of change in those values over the course of the session and the like.

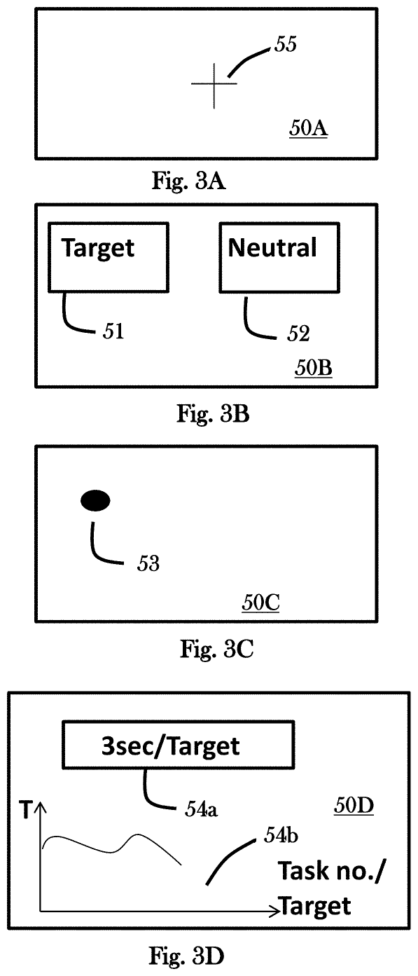

FIGS. 3A-3D visually illustrate the process of FIG. 2 by showing how some of the screens are represented throughout each task: FIG. 3A shows the gaze neutralizing screenshot 50A in which a cross 55 is presented at the center of the screen to neutralize/focus the subject's gaze. FIG. 3B shows another screenshot 50B in which the target and neutral images 51 and 52 respectively, are presented. FIG. 3C shows a screenshot 50C in which a probe 53 is shown replacing the target image 51 (to illustrate a congruent task type). FIG. 3d shows an optional screenshot 50D in which attentional feedback parameters are presented to the subject. In this example, two illustrative values are shown: a value reflecting real-time attentional bias delivered via real-time feedback at trial N Ti 54a (see FIG. 2 26); and additional form of post-session feedback indicative of some sort of aggregation of the subject's real-time attentional bias scores across the session or earlier trials represented as a graph or a histogram 54b showing the measured response time vs. the task number (See FIG. 2 27).

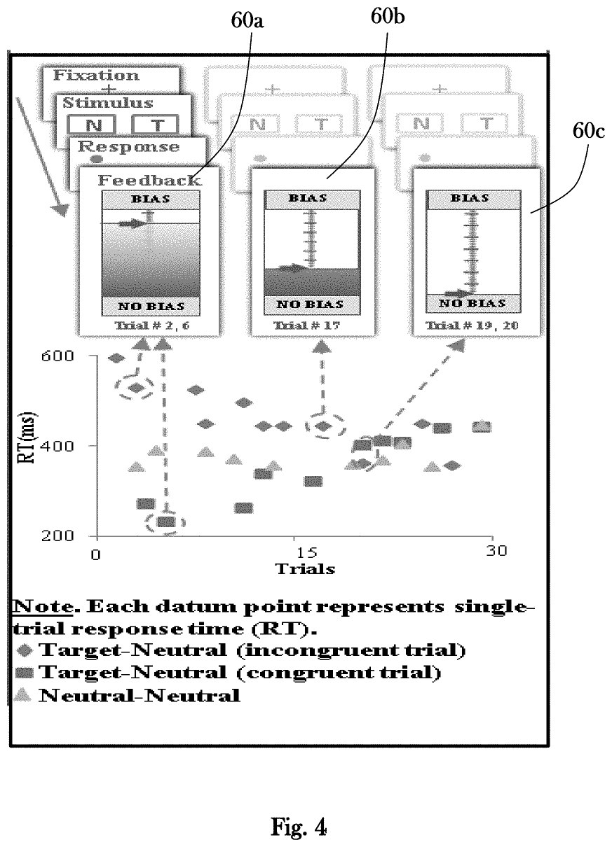

FIG. 4 also shows the steps of the process of FIG. 2 showing how the real-time attentional feedback can be presented through a colored scale showing real-time “bias level” on each trial N or visual feedback stimulus reflecting the calculated real-time attention bias score of the subject at each trial N. In this illustration and instantiation of the interference scheme (dot probe task) used to measure attentional bias, “high” of “low” real-time attentional bias levels or values of the subject in response to the current applied stimulus at trial N are graphically represented on the scale. FIG. 4 illustrates three bias levels per multiple trial N's: (i) a first high real-time attentional bias value/level 60a indicative of a delayed reaction time measured response on an incongruent trial (trial #2 60a) relative to a temporally contiguous preceding (N−1) neutral trial type and similarly high real-time attentional bias value/level 60a indicating in this illustration a fast reaction time measured responses on a congruent trial (trial #6 60a) relatively to a temporally contiguous preceding (N−1) neutral trial—reflecting momentary trial-level facilitated over-engagement towards the applied stimulus target or delayed disengagement from that target stimuli; (ii) a second bias level 60b wherein real-time attentional bias value/level indicative of a value/level somewhat lower than on trials #2 and #6 60a, calculated in the same fashion relative to a temporally contiguous preceding (N−1) neutral trial; and (iii) a third real-time attentional bias value/level 60c indicative of an absence of delayed (trial #19 60c) and an absence of a facilitated reaction time (trial #20 60c) measured response the an incongruent and congruent trials, respectively, relative to the temporally contiguous preceding (N−1) neutral trial—consistent with no or very low level of real-time attentional bias on these trials 60c as illustrated in the graph in FIG. 4.

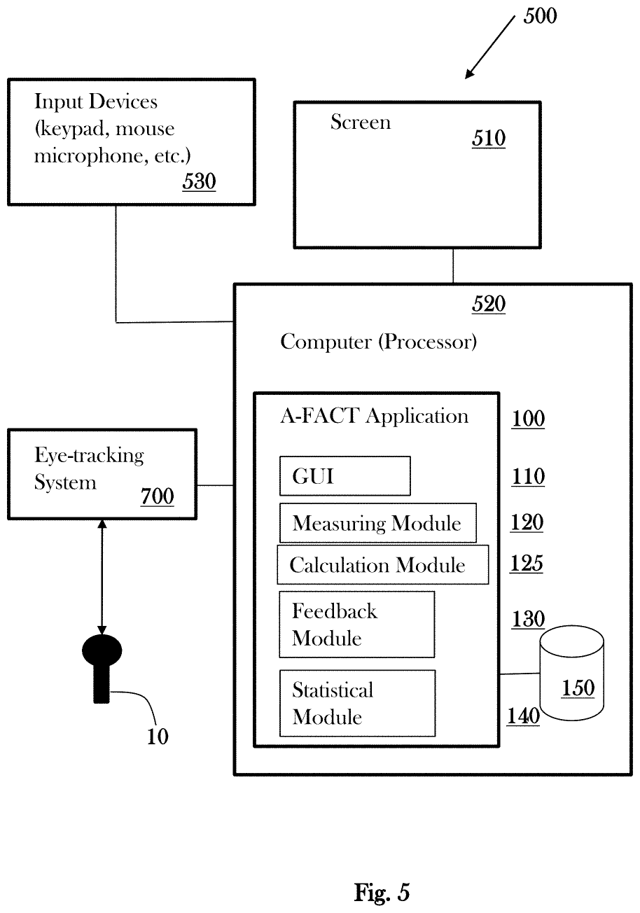

Reference is now made to FIG. 5, which schematically illustrates a system 500 for outputting real-time attentional feedback to the subject 10 indicative of the calculated real-time attentional bias score at each trial N, according to some embodiments of the present invention. As illustrated in FIG. 5, the system 500 includes Attention Feedback Awareness and Control Training (AFACT) application 100, which may include software and hardware components, at least one computer processor such as processor 520 enabling operating the AFACT application 100, receiving input data from various input devices and systems such as through standard computer input devices 530 e.g. a keypad, a microphone, a computer mouse, a touch screen, or any one or more other input device, and outputting devices such as a screen 510, a speaker (not shown) and the like.

Optionally, depending on system 500 definitions and AFACT application 100 capabilities, the system 500 further includes one or more stimuli and/or response measurement technology such as an eye-tracking system 700, as illustrated in FIG. 5 for either applying the sensory stimulus and/or measuring the subject's 10 measured response to the applied stimulus.

Any measuring device can be used to measure responses to an applied stimulus(i) on trial N that are observable indicators (physiological responses of the subject) of attentional bias of the subject that can then be used by the system to calculate in real-time an attentional bias score—so as to deliver real-time attentional feedback. The measuring device can be used to measure, for example, physiological response(s) of the subject to the applied stimulus on trial N and output data to the computerized system processor 520 indicative of the measured response to the stimulus for allowing the processor 520 to use this data to calculate in real-time the values of respective one or more attentional bias scores therefrom.

In this example an eye-tracking system 700 is used for measuring the subject's physiological (eye-movement), response to a visual stimuli which is an observable indicator of overt real-time attentional selection and modulation of the subject on trial N (time N) that can then be entered into the system and to which the attentional bias score calculation may be applied in real-time—so as to then deliver real-time attentional feedback. As known in the art, eye-tracking systems use laser technology to track the pupils of the subject 10 and therefore deduce the eyes' focal point, gaze duration and other such parameters as mentioned above. These parameters allow automatic measuring of the response time of the subject 10 to the visual stimulus. For example, when using the dot-probe, spatial cueing task and/or visual search task paradigms all involving using visual stimulus (images presented on-screen, the response to the visual stimulus in relation to the settings and conditions of the specific task can be automatically measured through the eye-tracking system 700 by locating the gaze of the subject 10 at each given moment and enabling to measure/calculate the time it takes the subject 10 to move his gaze from one location to another in response to the task requirements, for example. By integrating these eye-movement data and existing capacity of the eye-tracking technology into the system and applying in real-time attentional bias calculation to these data, we are able to use the eye-tracking technology to deliver an entirely novel capacity of real-time attentional feedback.

For example, consistent with norms, various observable physiological indicators from common eye-tracking systems may be recorded. For example, fixation may be operationalized as gaze within a radius of 30 pixels for at least 100-ms, averaged across both eyes, measured separately via corneal reflection. As another example, accompanying pupil size responses (dilation) can also be calculated from the eye-tracking system 700 measures to index degree of emotional arousal or cognitive effort concurrent with the dynamic moment-to-moment biased attentional processing of the target stimulus(i) (threat, smoking). This means that the measurements of the eye-tracking system allow deducing or extracting a variety of response types in addition to the response time measurement such as the aforementioned dilation parameter, which can indicate a psychological response to the stimulus.

According to some embodiments of the present invention, the AFACT application 100 may include one or more training programs each training program includes a set of tasks associated with a specific type of real-time attentional bias score and a predefined training method (e.g. dot-probe/visual search/spatial cueing—based attention allocation monitoring and feedback training program).

According to some embodiments of the present invention, as illustrated in FIG. 5, the AFACT application 100 includes a graphical user interface (GUI) 110, a measuring module 120, a calculation module 125, a feedback module 130, and a statistical module 140. The GUI 110 allows presenting data over the screen 510 and receiving input data from the various computer input devices 530 through a predefined GUI 110 platform. For example, the GUI 110 includes predefined input fields such as personal details fields—allowing the user to input details such as his/her age, weight, height, hobbies, variables relevant to bias or to the behaviors impacted by bias or attention allocation, and the like. The bias field may be a selection field requiring the subject 10 to select a bias type out of a predefined list (e.g. addiction type, threat type and the like). Each such field may be associated with a different training program allowing the subject 10 to be trained according to his/her special needs and type or expression of attentional bias to be calculated in real-time concurrent with its expression and trained via real-time attentional feedback. The computerized system can also select the bias type(s) and attentional feedback type for subject outcomes based on a variety of factors (e.g., calculated attentional bias scores, patterns or expression of such scores to various stimulus(i), information prior to delivery of feedback with respect to those or other cues). The GUI 110 may optionally also allow the subject 10 to select the stimulation type (e.g. auditory or visual) and/or the training goal (e.g., improve awareness and control of biased attentional processing of threatening or rewarding information/stimuli). Once the subject 10 or any other user such as a trainer inputs all or some of the fields such as personal details of the subject 10 and selects the bias type and training goal, the AFACT application 100 selects and retrieves a suitable training program associated with at least one of the details/parameters of these fields from one or more training programs databases such as database 150, which is stored in the memory of the computer 520.

According to some embodiments of the invention, the measuring module 120 enables measuring the measured responses indicative of attentional bias (e.g., physiological responses to the respective stimulus) of the subject 10 during each trial of each attentional feedback training session according to the layout of the system and the training program. For example, if using eye-tracking, the measuring module 120 enables communicating with the eye-tracking system 700 for receiving data related to measured gaze focusing and duration. The calculation module 125 receives the measured data from the measuring module 120 for calculating at least one attentional bias score associated with at least one attentional bias of the subject. The feedback module 130 enables the delivery of real-time feedback. The statistical module 140 further enables performing one or more diagnostic and related training exercises to the subject 10 such as assessing forms or patterns or levels of real time-time attentional bias, allowing each subject to receive real-time attentional feedback training on the attentional bias scores personalized to her/his biased attentional expression and respective needs.

According to some embodiments of the present invention, the feedback module 130 allows providing one or more feedback in real-time to the subject 10 indicative of the calculated real-time attentional bias score by, for instance, using a visualized bias scale, indicative of the degree and form and expression of that real-time attentional bias. The GUI 110 may allow the subject 10 or any other user to select and define the feedback presentation/output. For example, the visual presentation of the feedback may be a default while the GUI 110 allows the user/subject to select an additional/alternative channel for outputting the feedback such as through audio feedback. In this way both the visual representation of the feedback (e.g. response time number or response time performances histogram) and an audio output message announcing the response measures as the attention feedback can be provided to increase the impact (e.g., training impact) of the feedback over the subject 10 during training for increasing his/her awareness and thereby attentional control.

The statistical module 140 may enable recording of the calculated real-time attentional bias scores of the subject(s), and later use retrieval of these attentional bias data as well as related statistical data associated with each training program (e.g., personal details of the subject such as gender, age, nationality, religion and the like) to allow accumulating and, optionally, presenting statistical information that relates to the subject of attention allocation training. This may allow, for example, to keep records of training performances of each subject 10 (e.g., in relation to each specific bias type trained) and follow his/her awareness and attentional control. In this way the training application 100 may allow supporting a process of training and evaluation of its efficacy over time.

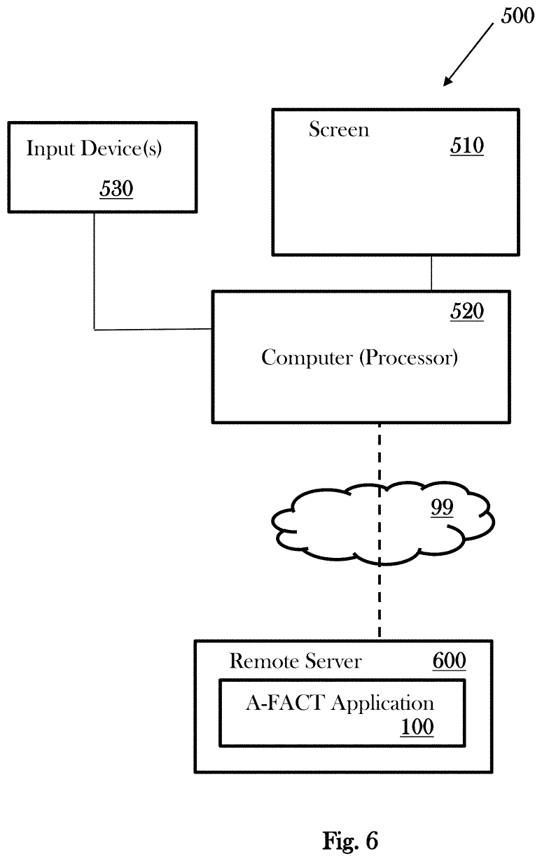

According to some embodiments of the present invention, as illustrated in FIG. 6, the AFACT application 100 may be operated through a remote server 600 communicating with client computers such as computer 520 through one or more communication links such as through an internet link 99 enabling thereby a multiplicity of end user to operate the AFACT application. This means that the AFACT application allows supporting operation of multiple training programs used by multiple subjects/user through their end computers or other electronic devices (e.g., smartphone, tablet device). In this case the application 100 may support an interactive website enabling subjects (users) to link thereto for training awareness and thereby control of biased attention, while the website allows recording training sessions' scores and various statistical evaluations and calculations to support a process of training and evaluation of its efficacy over time (much like FIG. 5 module 140).

The methods and systems of the present invention are expected to increase awareness to biases and afford the capacity to self-monitor those biases through learning a new association(s) enabled by real-time feedback, through AFACT training. Initially, an individual receives real-time feedback about her/his real-time attentional bias. A person thus learns a new association between (i) the events immediately preceding (e.g., exogenous or endogenous context or bias-facilitating cues such as thoughts, physical sensations, environmental context), the visual object(s) and other sensory stimuli to which attention is allocated, and immediately preceding biased/preferential allocation (e.g., thought, emotion, and/or physical cue) and (ii) the degree to which her/his attention was biased. This new learning occurs in real-time over the course of repeated trials via associative and operant conditioning. Elevated awareness of attentional bias is expected to lead to increased capacity of the subject to regulate or control attention and thereby reduce bias. Contemporary thinking regarding the primary mechanism underlying attentional bias, such as the inability to disengage attention (e.g., from threat cues), is driven by dysregulation in attentional control. Thus, the methods and systems of the present invention provide a novel means to train attention that will likely affect attentional bias by targeting the core mechanisms underlying bias.

Bias Awareness and Moderation of the Psychopathogenic Effects of Attentional Bias: In addition to facilitating increased control of attentional allocation, elevated awareness of bias is furthermore expected to moderate (buffer) the psychopathogenic effect(s) of attentional bias (e.g., drug-seeking in addiction maintenance, escape-avoidance in anxiety psychopathology). Thus, the proposed salutary moderation or buffering effect of attentional allocation awareness training as described above on potentially uncontrollable components of biased attentional allocation (e.g., threat detection underlying facilitated attention) is also expected to operate through an additional mechanism. Specifically, bias awareness and self-monitoring is expected to enable the capacity to engage in alternative behaviors to those typically driven by unmonitored attentional bias (e.g., behavioral choice, top-down behavioral inhibition in contrast to typically unmonitored, automatic pursuit of negative reinforcement opportunity), and thereby neutralize the typically automated psychopathogenic effects of attentional bias. Moreover, this additional salutary mechanism of feedback-facilitated awareness may, for some, serve as a “second-line of defense” in the event of transient attentional dyscontol. Finally, through increased awareness and capacity to self-monitor bias and to regulate and control attentional allocation, attention allocation awareness training is further expected to reduce the development and maintenance of multiple prevalent forms of psychopathology and addictions and therefore potentially serve as a prevention tool for preventing subjects from developing or enhancing psychopathology and/or addictions.

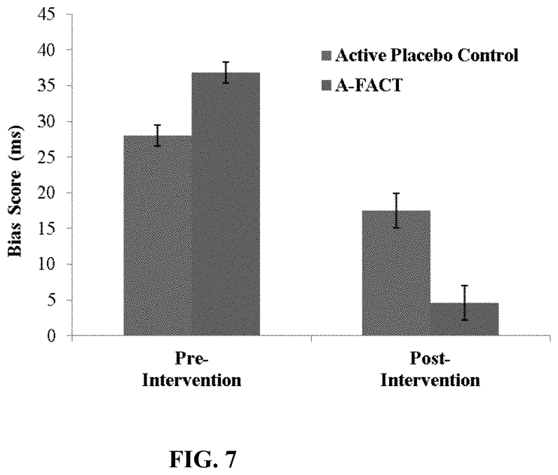

Reference is now made to FIG. 7, which shows experimental results of AFACT using the dot-probe paradigm for monitoring the attention allocation of a subject, providing real time feedback to the subject after each task in each training session. In a randomized control experimental design, we tested AFACT relative to an active placebo control condition among 40 anxious adult subjects demonstrating attentional bias to threat stimuli. The active placebo control condition was identical to the A-FACT condition except that in the former no real-time feedback was delivered to the subjects. Accordingly, in so far as AFACT engenders awareness-of-attention and thereby self-regulatory control, then bias reduction should result. Indeed, results shown in FIG. 7, demonstrated randomized control experimental evidence that AFACT ameliorates attentional bias.

Additional or alternative methods and paradigms can be used to calculate real-time attention bias scores and the four main paradigms mentioned above are but a few examples of existing methods and technologies that may be integrated within the system and to which the attentional bias score calculation methodology may be applied in real-time. As mentioned above, other types paradigms to present visual emotional or motivationally-relevant stimuli or information can be used and/or other types of sensory stimuli such as auditory and/or tactile stimuli and other types of observable indicators of attentional processing of said stimuli or information may be recorded in real time or near real time, inputted to the system and to which the real time attention bias index calculation methodology may be applied.

Additionally, or alternatively various types and techniques may be used to provide the feedback such as visual, auditory and the like. A combination of more than one type of feedback can be used.

Various technologies may be used to provide both real-time or near real-time feedback such as biofeedback and/or neurofeedback based technologies utilizing these technologies to provide feedback on the real-time attentional bias index.

For example in additional or alternative embodiments of the present invention, one or more devices that measure physiological responses to the applied stimulus may be used without requiring the subject to actually perform a task (beyond attending to applied stimulus(i)) to measure neurophysiological activation indicative of her attentional bias. For example a magnetic resonance imaging (MRI) device, EEG/ERP, etc. may be used to measure neurophysiological processes in real time when applying the stimulus (e.g., visual, tactile, auditory, olfactory and/or gustatory (taste) stimulus). As in earlier instantiations of this invention, the real-time feedback is presented to the subject substantially immediately after the stimulus is applied and the passive measured response to the stimulus is measured and calculated.

Many alterations and modifications may be made by those having ordinary skill in the art without departing from the spirit and scope of the invention. Therefore, it must be understood that the illustrated embodiment has been set forth only for the purposes of example and that it should not be taken as limiting the invention as defined by the following invention and its various embodiments and/or by the following claims. For example, notwithstanding the fact that the elements of a claim are set forth below in a certain combination, it must be expressly understood that the invention includes other combinations of fewer, more or different elements, which are disclosed in above even when not initially claimed in such combinations. The words used in this specification to describe the invention and its various embodiments are to be understood not only in the sense of their commonly defined meanings, but to include by special definition in this specification structure, material or acts beyond the scope of the commonly defined meanings. Thus if an element can be understood in the context of this specification as including more than one meaning, then its use in a claim must be understood as being generic to all possible meanings supported by the specification and by the word itself.

The definitions of the words or elements of the following claims are, therefore, defined in this specification to include not only the combination of elements which are literally set forth, but all equivalent structure, material or acts for performing substantially the same function in substantially the same way to obtain substantially the same result. In this sense it is therefore contemplated that an equivalent substitution of two or more elements may be made for any one of the elements in the claims below or that a single element may be substituted for two or more elements in a claim. Although elements may be described above as acting in certain combinations and even initially claimed as such, it is to be expressly understood that one or more elements from a claimed combination can in some cases be excised from the combination and that the claimed combination may be directed to a sub-combination or variation of a sub-combination.

Insubstantial changes from the claimed subject matter as viewed by a person with ordinary skill in the art, now known or later devised, are expressly contemplated as being equivalently within the scope of the claims. Therefore, obvious substitutions now or later known to one with ordinary skill in the art are defined to be within the scope of the defined elements.

The claims are thus to be understood to include what is specifically illustrated and described above, what is conceptually equivalent, what can be obviously substituted and also what essentially incorporates the essential idea of the invention.

Although the invention has been described in detail, nevertheless changes and modifications, which do not depart from the teachings of the present invention, will be evident to those skilled in the art. Such changes and modifications are deemed to come within the purview of the present invention and the appended claims.

### Appendix 1—MATLAB Code for Trial-Level Attentional Bias Calculation

The following MATLAB code shows a way to calculate attentional bias scores including various statistical operations on those scores.

### Code Use: Technical Instructions.

Input Preparation.

Input data needs to be organized in a single “long-file” of trial-level data (i.e., with one row per trial per participant). Rows need to be sorted, first, by subject number, and second, by ORIGINAL trial index reflecting the actual order trials were presented to each participant. Rows (trials) with error responses, or outlier response times (e.g., RT>1500 or RT<200; RT>3 SD from participants mean) need to be removed from the file before TL-BS analysis. Investigators are responsible for screening and cleaning data based on established practices for RT data and the unique features of their task design parameters.

Input Format.

See Table 1. Columns must be sorted as follows (the actual file must contain numbers only and no headers; see example input file attached), and saved as Excel (or .CSV file):

TABLE 1
Input
Data Format

| Column | 1 | 2 | 3 | 4 | 5 |
|---|---|---|---|---|---|
| Variable | Subject | Trial | Response | Congruency* | Block |
| | Number | Index | time (ms) | | Number** |
| Possible | 1 − N, | 1 − N | integer, > | {1, 2, 3} | 1 − N, |
| values | integer, > | integer, > | 0 | | integer, |
| | 0 | 0 | | | positive |

*Congruency: 1 = Incongruent trial, 2 = Congruent trial 3 = Neutral-Neutral trial.
**Block Number: If participants have a small break within the session, each segment (before and after the break) will get a consequent number (e.g., 1, 2 . . .). This is important to ensure that trials will not be matched between blocks/segments if doing so violates assumptions of time continuity between trials. If there was only one block, column #5 should contain ‘1’ throughout.

TABLE 2
Input Data Illustration

| | | | | |
|---|---|---|---|---|
| 101 | 1 | 340 | 1 | 1 |
| 101 | 2 | 565 | 1 | 1 |
| 101 | 3 | 490 | 3 | 2 |
| 101 | 4 | 480 | 2 | 2 |
| 101 | 5 | 210 | 1 | 2 |
| 102 | 1 | 970 | 2 | 1 |
| 102 | 2 | 840 | 2 | 1 |
| 102 | 3 | 510 | 3 | 1 |

Illustrative Input—Explanatory Note.

Here (Table 2) you can see a section of an input data table. The first participant (identified by the number ‘101’), has 5 trials (as indicated by 1-5 trial index column 3; for presentation purposes, usually there are many more trials per participant); a short break was taken after the first two trials, which is marked by the different block number (column 5).

Output Description.

The code produces two output files:

(1) Trial-Level Output Data.

An Excel file that contains the same table as in the input file, but with the addition of columns specifying TLBS computation per trial (first-level time-series data), in the event that such computations were possible. Table 3 below is an illustration of this output file format.

TABLE 3
Output Format - Trial-Level Data

| Column | 1-5 | 6 | 7 | 8 | 9 |
|---|---|---|---|---|---|
| Variable | [Input | TLBS | TLBS | TLBS | TLBS |
| | as | (IT − CT)* | (IT − CT) | (Neutral | (Neutral |
| | described | | Matching | Match)** | Match) |
| | above] | | Distance | | Matching |
| | | | | | Distan |

*IT − CT: This computation is done be matching and then subtracting congruent from incongruent trials' RT.
**Neutral Match: This computation is done be matching and then subtracting congruent or incongruent trials' RT from Neutral trials' RT. Selection of type of computation must be determined a priori based on conceptual aim analysis and various key methodological parameters of the task and data.

(2) TLBS Parameters Output Data.

An Excel file that contains the TL-BS parameters or features of the bias dynamics (i.e., second-level data/variables). Table 4 below is an illustration of this output file format for a single participant.

TABLE 4
Output Format - TLBS Parameters

| Column | 1 | 2 | 3 | 4 | 5 | 6 |
|---|---|---|---|---|---|---|
| Variable | SubjNum | traditionalBS | MeanTwd | MeanAwy | PeakTwd | PeakAwy |
| Description | Participant's | Aggregated | Mean | Mean | Peak | Peak |
| | number | mean | TLBS >= | TLBS <= | (maximum) | (minimum) |
| | | Bias | 0 | 0 | TLBS | TLBS |
| | | Score | | | towards | away |
| | | (IT-CT) | | | | |
| Column | 7 | 8 | 9 | 10 | 11 | 12 |
| Variable | Variability | MeanTwdNT | MeanAwyNT | PeakTwdNT | PeakAwyNT | VariabilityNT |
| Description | SD | Mean | Mean | Maximum | Minimum | SD(TLBS |
| | (TLBS) | TLBS | TLBS | TLBS | TLBS | Neutral |
| | | (Neutral | (Neutral | (Neutral | (Neutral | Match) |
| | | Match) >= | Match) <= | Match) | Match) | |
| | | 0 | 0 | towards | away | |
| Column | 13 | 14 | 15 | 16 | 17 | 18 |
| Variable | NumMatches | NumMatchesNT | MeanDist | MeanDistNT | SDDists | SDDistsNT |
| Description | Number of | Number of | Mean | Mean | SD of | SD of |
| | TLBS | TLBS | distance | distance | distances | distances |
| | values | values | between | between | between | between |
| | | (Neutral | TLBS | TLBS | matches | matches |
| | | Match) | matches | matches | | (Neutral |
| | | | | (Neutral | | Match) |
| | | | | Match) | | |

MATLAB Instructions for Code Use: Overview.

1. Place the attached files in a directory that Matlab can access.

2. Edit input/output file names in “MainTLBS.m”.

3. Run “MainTLBS.m”.

What is claimed is:

1. A computing system comprising:
at least one processor; and
at least one memory communicatively coupled to the at least one processor comprising computer-readable instructions that when executed by the at least one processor cause the computing system to implement a method of outputting a real-time attentional bias feedback, the method comprising:
a) applying, using at least one stimulation device, at least one sensory stimulus at time N over a human subject, said at least one sensory stimulus being associated with at least one attentional bias, wherein the at least one attentional bias represents impaired attentional engagement with or disengagement from the at least one sensory stimulus;
b) using at least one measuring device that measures physiological responses to the respective stimulus, measuring at least one response of the human subject to said applied at least one sensory stimulus at time N;
c) calculating at least one real-time attentional bias score of the subject at time N, wherein said at least one real-time attentional bias score is calculated based on: (1) the at least one measured response of the subject to said at least one applied sensory stimulus at time N; and (2) at least one temporally preceding measured response of the subject to the at least one applied sensory stimulus; and
d) using at least one output device, outputting in real-time the real-time attentional bias feedback at time N indicative of the calculated real-time attentional bias score at time N, said real-time attentional bias feedback being output relative to a corresponding attentional bias scale.

2. The computing system according to claim 1, wherein after steps a) to d) are executed initially, the system repeats steps b) to d) one or more times.

3. The computing system according to claim 1, wherein after steps a) to d) are executed initially, the system repeats steps a) to d) one or more times.

4. The computing system according to claim 1, wherein said at least one temporally preceding measured response comprises a temporally preceding adjacent measured response.

5. The computing system according to claim 1, wherein said at least one sensory stimulus comprises at least one of: visual stimulation, auditory stimulation, tactile stimulation, olfactory stimulation, and/or gustatory stimulation.

6. The computing system according to claim 1, wherein said measuring response in step b) comprises at least one of:
a) reaction time indicative of the time it took the subject to respond to the at least one applied stimulus at time N;
b) task time, indicative of the time it took the subject to fulfill a requested task associated with the respective applied stimulus at time N; or
c) at least one physiological measure indicative of physiological response of the subject to the stimulus at time N.

7. The computing system according to claim 1, wherein said measuring response in step b) is carried out by using at least one predefined attention interference scheme, and wherein said scheme is based on at least one of: a dot-probe paradigm; a spatial cueing paradigm; a visual search paradigm; and/or modified stroop task.

8. The computing system according to claim 1, wherein said measuring response in step b) is carried out by using an eye tracking system that measures the subject responses to visual stimuli.

9. The computing system according to claim 1, wherein said measuring device comprises one of: an eye-tracking system, a magnetic resonance imaging (MRI) device, a psychophysiological device, an electroencephalography (EEG) or an event-related potential (ERP) device.

10. The computing system according to claim 1, wherein said real-time attentional bias feedback is a visual feedback, auditory feedback, tactile feedback or any combination thereof.

11. The computing method according to claim 10, wherein said visual feedback comprises a graphical representation of the real-time bias level or form or characteristic(s) of the attentional bias of the subject calculated in real-time at time N.

12. The computing system according to claim 1, further comprising at least one input device for allowing the human subject to respond to said sensory stimulus_therethrough, wherein said input device includes a touch screen, a computer mouse, and a keypad.

13. The computing system according to claim 1, wherein the real-time attentional bias feedback is output in real-time for an entire duration when the at least one sensory stimulus is applied.

14. A computing system comprising:
at least one processor; and
at least one memory communicatively coupled to the at least one processor comprising computer-readable instructions that when executed by the at least one processor cause the computing system to implement a method of outputting a real-time attentional bias feedback, the method comprising:
a) applying, using at least one stimulation device, at least one sensory stimulus at time N over a human subject, said at least one sensory stimulus being associated with at least one attentional bias, wherein the at least one attentional bias represents impaired attentional engagement with or disengagement from the at least one sensory stimulus;
b) using at least one measuring device that measures physiological responses to the respective stimulus, measuring at least one response of the human subject to said applied at least one sensory stimulus at time N;
c) calculating at least one real-time attentional bias score, wherein said at least one attentional bias score is calculated based solely on the at least one measured response of the subject to said applied at least one sensory stimulus at time N; and
d) using at least one output device, outputting the real-time attentional bias feedback at time N indicative of the calculated real-time attentional bias score at time N, said real-time attentional bias feedback being output relative to a corresponding attentional bias scale.

15. A computing system comprising:
at least one processor; and
at least one memory communicatively coupled to the at least one processor;
at least one stimulation device configured to apply at least one sensory stimulus at time N over a human subject, said at least one sensory stimulus being associated with at least one attentional bias, wherein the at least one attentional bias represents impaired attentional engagement with or disengagement from a sensory stimulus;
at least one measuring device configured to measure at least one response of the human subject to said applied at least one sensory stimulus at time N; and
at least one output device configured to output a real-time attentional bias feedback at time N,
wherein the at least one processor is configured to calculate at least one real-time attentional bias score of the subject at time N,
wherein said at least one real-time attentional bias score is calculated based on: (1) the at least one measured response of the subject to said applied at least one sensory stimulus at time N;
and (2) at least one temporally preceding measured response of the subject to the applied at least one sensory stimulus; and
wherein the real-time attentional bias feedback is indicative of the calculated real-time attentional bias score at time N, said real-time attentional bias feedback being output relative to a corresponding attentional bias scale.
